(19) United States
(12) Patent Application Publication
(10) Pub. No.: US 2026/0048222 A1
(43) Pub. Date: Feb. 19, 2026

(54) FACEMASK HEADGEAR FRAMES

(71) Applicant: Sleepnet Corporation, Hampton, NH (US)

(72) Inventor: Jerry Gong, Andover, MA (US)

(21) Appl. No.: 18/805,859

(22) Filed: Aug. 15, 2024

(51) Int. Cl. A61M 16/06; A61M 16/08

(52) U.S. Cl. CPC A61M 16/0683; A61M 16/0622; A61M 16/0825

(57) ABSTRACT

Headgear frame members and facemask kits including the headgear frame members are described. A headgear frame member may include non-parallel elongated portions (e.g., branches) whose terminal end portions (including headgear connection points) are non-parallel to each other. In use, one terminal end portion of the headgear frame member may be positioned horizontally above the user's ear while the other terminal end portion may be positioned horizontally below the user's ear.

### BACKGROUND

Hospital and at home patients sometimes need supplemental respiratory support. Positive pressure ventilation, in which a supply of pressurized air is delivered to the user's airway, is often used. Positive pressure ventilation has been used to treat respiratory failure, respiratory insufficiency, and sleep apnea. There are a variety of user interfaces that can be used to provide positive pressure ventilation including nasal facemasks, full face facemasks, and nasal pillows for home and hospital use.

Positive airway pressure (PAP) is a treatment that uses air pressure to keep the airways open. PAP treatment typically is used by people who have breathing problems, such as sleep apnea. Sleep disorder breathing is a common disorder that causes pauses in breathing or shallow breaths while a person sleeps. As a result, not enough air reaches the person's lungs.

In obstructive sleep apnea, the person's airway collapses or is blocked during sleep. Air pressure from PAP treatment can prevent that person's airway from collapsing or becoming blocked.

PAP treatment generally involves: a PAP machine that blows therapeutic air and a PAP facemask assembly that receives the therapeutic air from the machine via an interconnecting hose. PAP facemask assemblies may comprise a facemask body of rigid or pliable (e.g., plastic) material, with a face-contacting sealing element (e.g., a silicone gasket seal or gel filled bladder), that is held in place with headgear (e.g., straps). The facemask body provides the structure for a headgear connector and straps that can be removably attached to the headgear connector. The scaling element provides a seal against the user's face, creating a chamber around the airway through which positive pressure ventilation can be applied.

U.S. Pat. No. 11,857,727 discloses a facemask frame including a single facemask connection (i.e., a central hole that fits over hardware, that is an elbow, for the facemask's air interface) and right and left arms each including non-parallel branches that terminate in strap connection points that are parallel to each other. Tilting activity of the facemask may be limited by tightening the straps with respect to the parallel connection points. However, the parallel nature of the connection points may require the straps be over tightened to make the single facemask connection effective.

### SUMMARY

A first aspect of the present disclosure relates to a headgear frame member, comprising: a first elongated portion having a first terminal end portion comprising a first coupling mechanism for removably coupling to headgear; a second elongated portion oriented non-parallel to the first elongated portion, the second elongated portion having a second terminal end portion comprising a second coupling mechanism for removably coupling to the headgear; and a third elongated portion oriented non-parallel to the first elongated portion and the second elongated portion, wherein the third elongated portion has an axis extending along the third elongated portion, wherein the first elongated portion extends a first distance along the axis, wherein the second elongated portion extends a second distance along the axis, and wherein the first distance is different than the second distance.

In some embodiments of the first aspect, the first distance is greater than the second distance.

In some embodiments of the first aspect, the third elongated portion has a third terminal end portion, the first coupling mechanism is a third distance away from the third terminal end portion along the axis, and the second coupling mechanism is a fourth distance away from the third terminal end portion along the axis.

In some embodiments of the first aspect, the third distance is greater than the fourth distance.

In some embodiments of the first aspect, the third terminal end portion comprises a third coupling mechanism, the first coupling mechanism is the third distance away from the third coupling mechanism along the axis, and the second coupling mechanism is the fourth distance away from the third coupling mechanism along the axis.

In some embodiments of the first aspect, the third distance is greater than the fourth distance.

In some embodiments of the first aspect, the first coupling mechanism comprises a first elongated aperture having a first major axis, the second coupling mechanism comprises a second elongated aperture having a second major axis, and the first major axis and the second major axis intersect at a location between the headgear frame member and the back of the user's head.

In some embodiments of the first aspect, the first coupling mechanism removably couples to the headgear above an ear of a user and the second coupling mechanism removably couples to the headgear below the ear of the user.

In some embodiments of the first aspect, the third elongated portion has a third terminal end portion comprising a third coupling mechanism for removably coupling to another headgear frame member.

In some embodiments of the first aspect, the third coupling mechanism comprises a hook member sized to removably couple to the perimeter of an aperture in the other headgear frame member.

In some embodiments of the first aspect, the first and second elongated portions are positioned at an angle of about 60° to 90° with respect to each other.

In some embodiments of the first aspect, the angle is about 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, or 90°.

In some embodiments of the first aspect, the angle is about 80°.

In some embodiments of the first aspect, when the hypotenuse of a right triangle is positioned along a portion of the headgear removably coupled to the first elongated portion, the interior angle of the corner of the right triangle abutting the portion of the headgear is about 25° to 55°, and the interior angle of the corner of the right triangle abutting the first elongated portion is about 35° to 65°.

In some embodiments of the first aspect, the interior angle of the corner of the right triangle abutting the portion of the headgear is about 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, or 55°.

In some embodiments of the first aspect, the interior angle of the corner of the right triangle abutting the portion of the headgear is about 40°.

In some embodiments of the first aspect, the interior angle of the corner of the right triangle abutting the first elongated portion is about 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, or 65°.

In some embodiments of the first aspect, the interior angle of the corner of the right triangle abutting the first elongated portion is about 50°.

A second aspect of the present disclosure relates to a facemask kit, comprising: a headgear frame member of the first aspect and headgear comprising a first elongated member and a second elongated member, wherein the first elongated member of the headgear removably couples to the first elongated portion of the first frame member and adjustment of the tension of the first elongated member alters the force exerted perpendicularly with respect to a sealing element of the facemask, and wherein the second elongated member of the headgear removably couples to the second elongated portion of the first frame member and adjustment of the tension of the second elongated member vertically raises and lowers the positioning of the facemask on the user's face.

In some embodiments of the second aspect, the facemask kit comprises a second headgear frame member comprising: a third elongated portion having a third terminal end portion comprising a third coupling mechanism for removably coupling to the headgear; a fourth elongated portion oriented non-parallel to the third elongated portion, the fourth elongated portion having a fourth terminal end portion comprising a fourth coupling mechanism for removably coupling to the headgear; and a fifth elongated portion oriented non-parallel to the third elongated portion and the fourth elongated portion, wherein the fifth elongated portion has a second axis extending along the fifth elongated portion, wherein the third elongated portion extends the first distance along the second axis, and wherein the fourth elongated portion extends the second distance along the second axis.

In some embodiments of the second aspect, the facemask kit comprises a third headgear frame member that removably couples to the headgear frame member and the second headgear frame member.

In some embodiments of the second aspect, the facemask kit comprises a facemask body that removably couples to the third headgear frame member.

In some embodiments of the second aspect, the facemask body comprises a fluid-filled cushion that facilitates a seal between the facemask body and the user's face.

### BRIEF DESCRIPTION OF DRAWINGS

For a more complete understanding of the present disclosure, reference is now made to the following description taken in conjunction with the accompanying drawings.

FIG. 1 is a deconstructed planar view of a headgear and headgear frame members.

FIG. 2A is an elevated front perspective view of a PAP facemask assembly.

FIG. 2B is a front view of the PAP facemask assembly of FIG. 2A.

FIG. 2C is a back view of the PAP facemask assembly of FIG. 2A.

### DETAILED DESCRIPTION

The present disclosure provides, among other things, headgear frame members and facemask kits including the headgear frame members.

A headgear frame member of the present disclosure includes non-parallel elongated portions (e.g., branches) whose terminal end portions (including headgear connection points) are vertically non-parallel to each other. In use, one terminal end portion of the headgear frame member may be positioned horizontally above the user's ear while the other terminal end portion may be positioned horizontally below the user's ear. Each terminal end portion may removably couple to a different strap of a headgear.

A headgear frame member of the present disclosure may further include a third elongated portion (e.g., branch) that is oriented non-parallelly with respect to the other elongated portions of the headgear frame member. The third elongated portion includes a mechanism for removably coupling the headgear frame member to another member of the headgear frame that in turn removably couples to a facemask body.

The use of independent vertically non-parallel terminal end portions that each removably couple to headgear supports a more robust contact between the facemask's sealing element and the user's face as compared to headgear frame members having vertically parallel terminal end portions.

A user's face may have side-to-side variations/asymmetries in appearance and/or muscularity, for example due to indentations from surgeries, muscular degeneration from stroke or bell's palsy, and swelling due to injury or allergy. Side-by-side variations can compromise facemask fit. The use of vertically non-parallel terminal end portions in a headgear frame member of the present disclosure allows for a facemask of the present disclosure to sufficiently seal to the user's face despite side-to-side variations.

The vertically non-parallel terminal end portions afford the ability to make fine-tuned vertical and horizontal adjustments using independent headgear portions (e.g., straps). This is opposed to the only horizontal adjustments permitted via use of vertically parallel terminal end portions.

Moreover, the vertically non-parallel terminal end portions support elongation, as compared to the compression and wrinkling resulting from use of vertically parallel terminal end portions. The non-parallel portions of a headgear frame member of the present disclosure may pull at an angle that stretches the Y axis of the facemask (e.g., increases the vertical contact surface of the facemask sealing element to the user's face). By comparison, vertically parallel terminal end portions compress the facemask in with no change in Y axis, which ultimately wrinkles the facemask if pulled to a sufficient tension.

Anchors, or strain relief supports, on the sealing element perpendicular to the sealing surface, may further ground sections of the facemask, thereby enhancing the elongation when tension is applied by the headgear. These anchors exist as structures perpendicular to the sealing surface of the mask so when non-parallel tension of the headgear is applied the base tends to stay in place while the mask's sealing surface stretches, causing the anchor structure to lean away from perpendicular in a direction that is dependent on the tensions applied.

### Headgear Frames

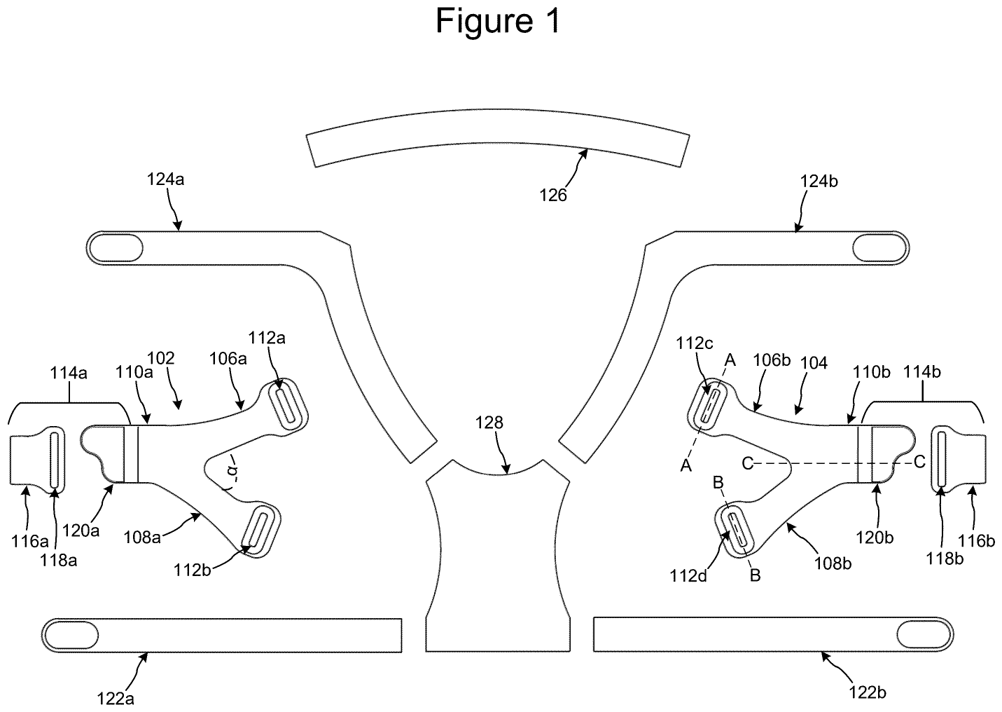

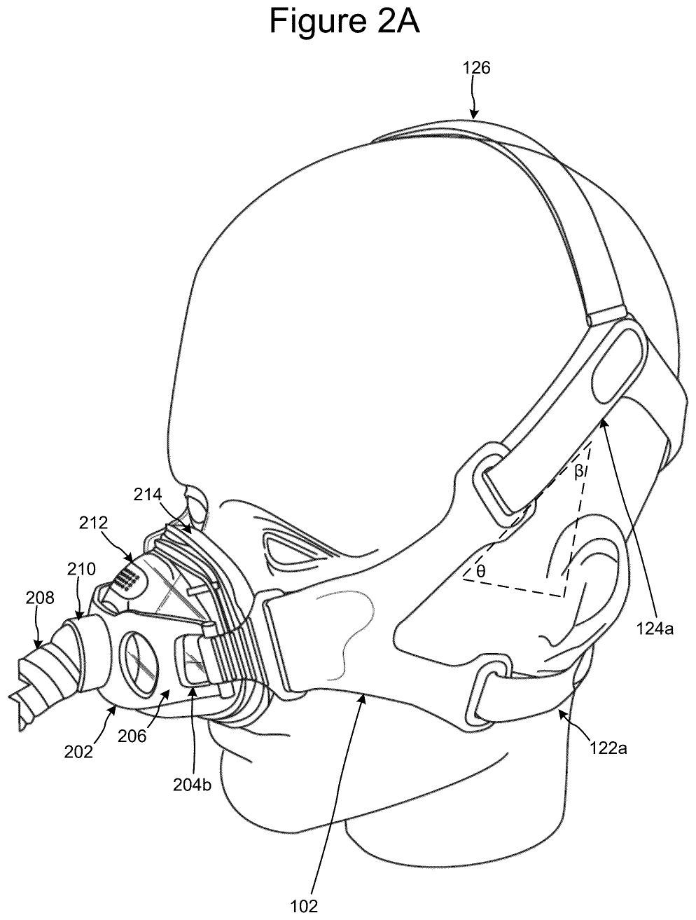

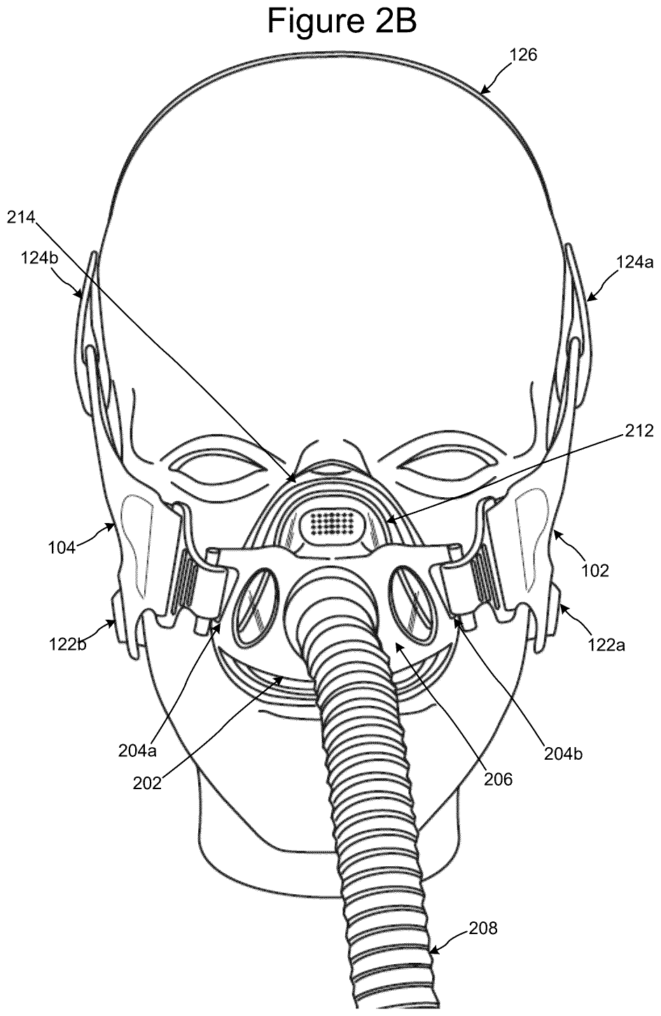

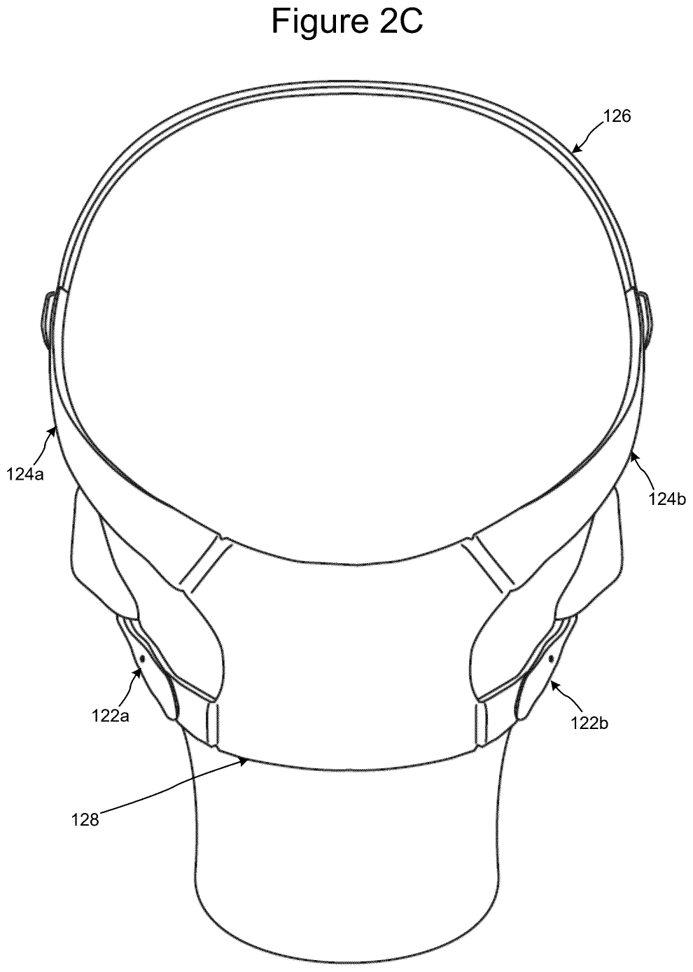

A headgear frame of the present disclosure may include three frame members: a first frame member 102 (illustrated in FIGS. 1-2B); a second frame member 104 (illustrated in FIGS. 1 and 2B); and a third frame member 202 (illustrated in FIGS. 2A-2B).

The first and second frame members 102/104 may each include a first elongated portion 106, a second elongated portion 108, and a third elongated portion 110. The first, second, and third elongated portions 106/108/110 may all extend away from each other such that each elongated portion extends in a non-parallel direction with respect to each of the other elongated portions. That is, the first elongated portion 106 may extend in a non-parallel direction with respect to the second and third elongated portions 108/110, the second elongated portion 108 may extend in a non-parallel direction with respect to the first and third elongated portions 106/110, and the third elongated portion 110 may extend in a non-parallel direction with respect to the first and second elongated portions 106/108.

The first and second elongated portions 106/108 may each have a terminal end portion including a coupling mechanism 112 for removably coupling to headgear. For example, the terminal end portion of the first elongated portion 106a (of the first frame member 102) may include a coupling mechanism 112a, the terminal end portion of the second elongated portion 108a (of the first frame member 102) may include a coupling mechanism 112b, the terminal end portion of the first elongated portion 106b (of the second frame member 104) may include a coupling mechanism 112c, and the terminal end portion of the second elongated portion 108b (of the second frame member 104) may include a coupling mechanism 112d. When in use, the coupling mechanism 112, of the first elongated portion 106, may removably couple to a strap of headgear horizontally above an car of the user and the coupling mechanism 112, of the second elongated portion 108, may removably couple to another strap of the headgear horizontally below the user's car.

As illustrated, the coupling mechanisms 112 of a frame member may be the same. However, the present disclosure envisions situations where the coupling mechanisms 112 of a single frame member are different.

Moreover, as illustrated the coupling mechanism 112 may include an aperture for receiving a strap of headgear. A headgear strap may insert through the aperture of the coupling mechanism 112 and then be removably coupled (e.g., via hook and loop fastener) to a section of the headgear (e.g., another section of the strap). The present disclosure envisions different coupling mechanisms than that illustrated. The present disclosure encompasses all coupling mechanisms usable to removably couple the first and second frame members 102/104 to headgear.

In some embodiments, the coupling mechanisms 112 of the first and second elongated portions 106/108 may include elongated apertures. The elongated aperture of the first elongated portion 106 may have a first major axis (see line A-A in FIG. 1), the elongated aperture of the second elongated portion 108 may have a second major axis (see line B-B in FIG. 1), and the major axes may be non-parallelly oriented. As can be deduced from FIG. 1, in some embodiments, the first and second major axes may intersect at a location between the frame member 102/104 and a back of the user's head (i.e., the first and second major axes may intersect at a location behind the frame member 102/104). This orientation of the major axes allows a user to adjust the tension of the headgear straps engaging the first elongated portions 106a/106b to vertically raise and lower the positioning of the facemask with respect to an initial positioning of the facemask on the user's face. In other words, increasing the tension of the headgear straps engaging the first elongated portions 106a/106b may elongate the contact surface of the facemask sealing element on the user's face, which may increase the seal between the user's face and the sealing element of the mask body. The user may adjust the tension of the headgear straps engaging the second elongated portions 108a/108b to adjust the force exerted perpendicularly with respect to the sealing element of the mask body.

The third elongated portion 110 has a terminal end portion including a coupling mechanism 114 for removably coupling the frame member 102/104 to the third frame member 202 (i.e., another frame member). For example, the terminal end portion of the third elongated portion 110a (of the first frame member 102) may include a coupling mechanism 114a and the terminal end portion of the third elongated portion 110b (of the second frame member 104) may include a coupling mechanism 114b.

As illustrated, the coupling mechanism 114 may include a hook member 116 sized to removably couple to the perimeter of an aperture 204 (illustrated in FIGS. 2A-2B) in the third frame member 202. The hook member 116 may include an aperture 118 for receiving a portion 120 of the third elongated portion 110. The portion 120 may insert through the aperture 118 and then be removably coupled (e.g., via hook and loop fastener) to a section of the frame member 102/104. The present disclosure envisions different coupling mechanisms. The present disclosure encompasses all coupling mechanisms usable to removably couple the third elongated portion 110 to the third frame member 202.

The third elongated portion 110 may have an axis (see line C-C in FIG. 1) that extends along the third elongated portion 110. The first and second elongated portions 106/108 may extend different distances along the axis. That is, the first elongated portion 106 may extend a first distance along the axis while the second elongated portion 108 may extend a second distance along the axis. In some embodiments, the first elongated portion 106 may extend a farther distance along the axis than the second elongated portion 108; that is the abovementioned first distance may be greater than the abovementioned second distance (see FIG. 1). Put another way, the terminal end portion/coupling mechanism 112 of the first elongated portion 106 may be a first distance away from the terminal end portion/coupling mechanism 114 of the third elongated portion 110 along the axis, the terminal end portion/coupling mechanism 112 of the second elongated portion 106 may be a second distance away from the terminal end portion/coupling mechanism 114 of the third elongated portion 110 along the axis, and, in some embodiments, the first distance may be greater than the second distance.

The third frame member 202 may include a convex surface 206 and a corresponding concave surface. In at least some embodiments, the concave surface may have a concavity commensurate with a convexity of an outer convex surface of the facemask body to which it is to removably couple or is permanently coupled to.

The third frame member 202 may include one or more apertures within and extending between the convex surface 206 and the concave surface. As illustrated, the third frame member 202 may include a first aperture 204a and a second aperture 204b. The first aperture 204a may be configured to receive a portion of the coupling mechanism 114b of the second frame member 104 and the second aperture 204b may be configured to receive a portion of the coupling mechanism 114a of the first frame member 102. The coupling mechanism 114b and the coupling mechanism 114a may act upon the edge perimeters of the first aperture 204a and the second aperture 204b, respectively, to removably couple the first and second frame members 102/104 to the third frame member 202.

The size, shape, and placement of an aperture 204 in the third frame member 202 is not limited to that illustrated. The size, shape, and placement of an aperture 204 are configurable, provided that the aperture 204 is configured to receive a portion of a coupling mechanism 114 and be acted thereupon to removably couple the first and second frame members 102/104 to the third frame member 202. For example, in some embodiments an aperture 204 may be shaped to correspond with a shape of the hook member 116.

In some embodiments, instead of being configured with one or more apertures 204, the third frame member 202 may include one or more indentations in the concave surface 206, and optionally corresponding one or more protrusions extending from the convex surface. In such embodiments, the one or more indentations may be configured to receive the one or more hook members 116, respectively, to removably couple the first and second frame members 102/104 to the third frame member 202.

The removable coupling of the third frame member 202 to the first and second frame members 102/104 is not intended to be limited to that illustrated. For example, the coupling mechanism 114 may not include the hook member 116. Instead, the portion 120 may pass through the aperture 204 and thereafter be removably coupled (e.g., via hook and loop fastener) to a section of the headgear frame member 102/104 it is a part of.

The third frame member 202 may be configured to receive and connect to a tube 208 configured to provide pressurized air. In at least some embodiments, the third frame member 202 may include a ball and socket connector 210 configured to receive and provide a seal with the tube 208. The ball and socket connector 210 is a coupling mechanism that includes a spherical socket configured to receive a corresponding spherical end of the tube 208. In some embodiments, the end of the tube 208 may be semi-spherical but nonetheless capable of providing a seal with the walls of the spherical socket of the ball and socket connector 210. A spherical end of the tube 210 and a semi-spherical end of the tube 210 may each be referred to as a ball portion of the tube 210 herein. The spherical nature of the ball and socket connector 210, combined with the spherical (or semi-spherical) end of the tube 208, allows for multidirectional movement and rotation of the tube 208 within the ball and socket connector 210. Such multidirectional movement permits a user to take up different positions with respect to a PAP compressor/motor without experiencing tube drag, or with experiencing less tube drag than other known PAP configurations.

In at least some embodiments, the ball and socket connector 210 may include an elongated portion configured to be inserted through an aperture in a facemask body when the facemask body is removably or permanently coupled to the third frame member 202. In at least some embodiments, a seal between the tube 208 and the chamber of a facemask body may be provided solely by the elongated portion, of the ball and socket connector 210, having an outer perimeter commensurate with a perimeter wall of the aperture of the facemask body. In such embodiments, the size and shape of the outer perimeter, of the elongated portion of the ball and socket connector 210, and the perimeter wall of the aperture of the facemask body are configurable provided they are sufficiently similar to provide a seal.

The first, second, and third frame members 102/104/202 may be constructed using a thermoplastic polymer. In some embodiments, the thermoplastic polymer may be polypropylene. The first, second, and third frame members 102/104/202 may additionally or alternatively be constructed using one or more rather flexible materials, such as PVC and/or acrylonitrile-butadiene-styrene (ABS). The first, second, and third frame members 102/104/202 may additionally or alternative be construed using one or more malleable metals, which may bend under applied force and retain form adjustments.

While the above describes the first, second, and third frame members 102/104/202 being separate pieces of a headgear frame, in some embodiments, the first, second, and third frame members 102/104/202 may be a singular structure such that the first and second frame members 102/104 are permanently attached to the third frame member 202.

The first, second, and third elongated portions 106/108/110 may be oriented at various angles with respect to each other. The first and second elongated portions 106/108 may be positioned at an angle α (illustrated in FIG. 1) with respect to each other. In some embodiments, angle α may be about 60° to 90°. In some embodiments, angle α is about 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, or 90°. In some embodiments, angle α is about 80°. Angle α of the first frame member 102 may be the same or different from angle α of the second frame member 104.

Moreover, the coupling mechanism 112a/112c may be oriented to removably couple to the headgear at different angles. As illustrated in FIG. 2A, a right triangle may be positioned such that its hypotenuse is positioned along the portion of the headgear removably coupled to the coupling mechanism 112a/112c. The angles β and θ may be varied, provided that the sum of angles β and θ is 90°. Angles β and θ of the first frame member 102 may be the same or different from angles β and θ of the second frame member 104.

In some embodiments, angle β may be about 25° to 55°. In some embodiments, angle β is about 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, or 55°. In some embodiments, angle β is about 40°.

In some embodiments, angle θ may be about 35° to 65°. In some embodiments, angle θ is about 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, or 65°. In some embodiments, angle θ is about 50°.

### Headgear

The present disclosure envisions utilization of any headgear configuration capable of being used in conjunction with the herein described first and second frame members 102/104. An illustrative headgear is illustrated in FIGS. 2A-2C, with a deconstructed view of the headgear being provided in FIG. 1.

The headgear may include first and second elongated members (i.e., straps) 122a/122b. The first elongated member 122a may removably couple to the coupling mechanism 112b, of the first frame member 102, such that the first elongated member 122a is positioned below the user's left car in use. Likewise, the second elongated member 122b may removably couple to the coupling mechanism 112d, of the second frame member 104, such that the second elongated member 122b is positioned below the user's right ear in use.

As an example, the illustrated first and second elongated members 122a/122b may pass through the apertures of the coupling mechanisms 112b/112d and then be removably coupled (e.g., via hook and loop fastener) to a section of the headgear (e.g., another section of the corresponding elongated member). However, the present disclosure envisions different coupling mechanisms than that illustrated. The present disclosure encompasses all coupling mechanisms usable to removably couple the first and second elongated members 122a/122b to the corresponding coupling mechanisms 112b/112d.

The headgear may further include third and fourth elongated members (i.e., straps) 124a/124b. The third elongated member 124a may removably couple to the coupling mechanism 112a, of the first frame member 102, such that the third elongated member 124a is positioned above the user's left ear in use. Likewise, the fourth elongated member 124b may removably couple to the coupling mechanism 112c, of the second frame member 104, such that the fourth elongated member 124b is positioned above the user's right ear in use.

As an example, the illustrated third and fourth elongated members 124a/124b may pass through the apertures of the coupling mechanisms 112a/112c and then be removably coupled (e.g., via hook and loop fastener) to a section of the headgear (e.g., another section of the corresponding elongated member). However, the present disclosure envisions different coupling mechanisms than that illustrated. The present disclosure encompasses all coupling mechanisms usable to removably couple the third and fourth elongated members 124a/124b to the corresponding coupling mechanisms 112a/112c.

The headgear may further include a fifth elongated member (i.e., strap) 126 that may be permanently coupled (e.g., via stitching) to the third and fourth elongated members 124a/124b. In use, the fifth elongated member 126 may extend across the top of the user's head.

The headgear may further include a main member 128 that is positioned against the back of the user's head in use. Each of the first, second, third, and fourth elongated members 122a/122b/124a/124b may be permanently coupled (e.g., via stitching) to and extend from the main member 128.

The members of the headgear may be composed of one or more elastic materials to facilitate user comfort. However, the elasticity may be selected such that the headgear is not too elastic to comprise the seal of the facemask to the user's face. Example materials that the headgear may be made of include spandex, high density polyurethane foam, cotton, silk, wool, polyester, nylon, rayon, and other woven fabric materials.

### Facemask Bodies

FIGS. 2A through 2B illustrate an example facemask body 212 of the present disclosure. The facemask body has a cushion 214 removably or permanently coupled thereto. In some embodiments, the facemask body 212 may be configured to fit over at least a portion of a nose (e.g., at least nostrils) of a user. Such may be referred to as a nasal facemask body. In some other embodiments, the facemask body 212 may be configured to fit over a mouth and at least a portion of a nose of a user. Such may be referred to as an oronasal facemask body. In some embodiments, the facemask body 212 may be low-profile, meaning the facemask body 212 may fit over at least a portion of the user's nose, but not extend all the way up to the bridge of the user's nose. Such a facemask body 212 may be more trapezoidal than triangular.

The facemask body 212 includes an inner concave surface defining a chamber. The dimensions of the inner concave surface, and thus the volume of the chamber, are configurable based on nose heights of users and/or air volume requirements for proper (e.g., PAP) use. The facemask body 212 includes an outer convex surface corresponding to the inner concave surface.

The facemask body 212 includes an aperture located through the outer convex surface and the inner concave surface. The aperture may have a continuous perimeter extending between the outer convex surface and the inner concave surface, where the continuous perimeter is configured to create a seal (e.g., an airtight seal) with a portion of the third frame member 202. The size, shape, and placement of the aperture are configurable, provided that the aperture may receive and create a seal with a portion of the third frame member 202.

The facemask body 212 may be retained against the third frame member 202 solely by an extension element of the third frame member 202 being positioned within the aperture of the facemask body 212, and the continuous perimeter of the aperture friction fitting against the extension element. This friction fitting of may be the only mechanism by which the facemask body 212 is retained against the third frame member 202.

In some embodiments, the facemask body 212 may include a retaining element (not illustrated) (e.g., a wire) configured to enable a user to form the outer convex surface and the inner concave surface, and thus the chamber of the facemask body 212, to features/contours of the user's face, thereby enabling a better seal of the facemask body 212 to the user's face. In some embodiments, the facemask body 212 may not include the retaining element. For example, in pediatric applications a sufficient seal may be provided via tension applied on the facemask body 212 by the headgear.

In some embodiments, the retaining element may be disposed within the facemask body 212 between the outer convex surface and the inner concave surface. In other embodiments, the retaining element may be affixed to the outer convex surface. In still other embodiments, the retaining element may be affixed to the inner concave surface.

In embodiments where the facemask body 212 is configured to surround a mouth of a user, the retaining element may include a portion configured to be positioned along a chin of the user. In some embodiments, this portion of the retaining element may be stiffened, for example by molding a polycarbonate sleeve over the portion of the retaining element before the retaining element, and polycarbonate sleeve, are molded in or affixed to the facemask body 212. Stiffening of this portion of the retaining element causes the portion to become more difficult to bend but not to be rigid (i.e., to be stiffened compared to the remainder of the retaining element).

It is noted that there are a number of means known to those skilled in the art to stiffen the aforementioned portion of the retaining element. A polycarbonate overmold is one of the least costly and most aesthetically attractive. It is also noted that the portion of the retaining element that is stiffened may nonetheless still have some “give” in it.

The retaining element can be constructed from any suitable material that is malleable and capable of retaining a shape into which it is formed. In some embodiments, the retaining element may be constructed from one or more malleable metal wires or strips. In some embodiments, the retaining element may be constructed using one or more stainless steel wires or strips. In some embodiments, the retaining element may be constructed from one or more non-metal materials, such as polypropylene or some other non-metal material that is capable of retaining its shape as it is bent.

The facemask body 212 additionally includes a cushion attachment surface configured to have the cushion 214 affixed thereto. Details of the cushion 214 are described herein below.

In at least some embodiments, the facemask body 212 may be formed from a single material. In at least some embodiments, the facemask body 212 may be formed from a single flexible material. More specifically, in at least some embodiments the inner concave surface, the outer convex surface, and the cushion attachment surface may all be formed from the same flexible material. In some embodiments, the flexible material may be a malleable plastic. In some embodiments, the malleable plastic may be polyvinyl chloride (PVC).

### Facemask Cushions

The cushion 214 may be removably or permanently coupled to the cushion attachment surface of the facemask body 212. In some embodiments, the cushion 214 may be permanently adhered (e.g., using glue or another adhesive) to the cushion attachment surface.

The cushion 214 can be comprised of one or more formed flexible materials having a durometer value of 60 or less, 50 or less, 40 or less, 30 or less, 20 or less, or 10 or less on a Type 000 Shore scale.

In some embodiments, the cushion 214 can be comprised of one or more formed flexible materials having a durometer value of about 60 to about 90 on Type 000 Shore scale. In some embodiments, the formed flexible materials may have a durometer value of about 60, about 65, about 70, about 75, about 80, about 85, or about 90 on the Type 000 Shore scale.

Example cushions of the present disclosure are silicone gel bladders, air-filled bladders, foam-filled bladders, liquid rubber-filled bladders, molded foam and molded rubber.

The Shore durometer is a device known to one of ordinary skill in the art for measuring the hardness of a material, typically of polymers, elastomers, and rubbers. Higher numbers on the scale indicate a greater resistance to indentation and thus harder materials. Lower numbers indicate less resistance and softer materials. The term is also used to describe a material's rating on the scale, as in an object having a “Shore durometer of 90.” Shore durometers are designed to measure specific scale ranges. A Type 000 durometer measures the hardness of very soft materials.

By configuring the cushion 214 to have a durometer value of 60 or less on a Type 000 Shore scale, the facemask body 212 may contour to the user's face as it experiences tension from headgear, and the cushion 214 may fill in the gaps not accommodated by pure contouring of the facemask body 212. The cushion 214 does this while maintaining a thin profile and reasonable comfort for the user.

The cushion 214 may include an external membrane bladder containing a fluid, for example a gel, for example a silicone gel. The external membrane bladder may be formed from one or more thermopolymer sheets. Thermopolymer sheet material is known to one of ordinary skill in the art. Examples of thermopolymer sheet materials include those comprising urethane, polyurethane, latex, nitrile, and other natural and synthetic polymers. The external membrane bladder may be formed from a sheet of thermopolymer comprising, for example, first and second thermopolymer sheet portions. The term “thermopolymer sheet portion” is defined to mean a portion of a single sheet folded or otherwise manipulated to form more than one face of the external membrane bladder.

In some embodiments, gel (e.g., silicone gel) may be used to fill the external membrane bladder. According to the present disclosure, a cushion 214, having an external membrane bladder filled with gel, may have a durometer value of 60 or less, 50 or less, 40 or less, 30 or less, 20 or less, or 10 or less on a Type 000 Shore scale. However, it is noted that the present disclosure envisions materials, other than gel, being used to fill the external membrane bladder while still maintaining a durometer value of 60 or less on a Type 000 Shore scale.

The cushion 214 may be triangular in cross-section with three primary external faces. The cushion 214 may include a skin contact face, an attachment face attached to the attachment surface of the facemask body 212, and a third face. In cross-section, the skin contact face may be the longest face. The attachment face can be longer than, shorter than or equal in length to the third face. In some embodiments, the third face has a length less than about 70% to 90% of the length of the skin contact face. The angle formed between the attachment face and the third face may be between 70 degrees and 90 degrees.

The third face may occupy the inner perimeter of the cushion 214 and the skin contact face may occupy the outer perimeter of the cushion 214. In cross-section, the transition between the faces of the cushion 214, particularly the transition between the third face and the skin contact face, may appear rounded, slightly rounded, or even flat.

In some embodiments, the cushion 214 can have more than three primary faces. One skilled in the art will recognize, for example, that a trapezoidal seal will offer many of the advantages described herein in connection with a triangular cushion, and one skilled in the art, guided by the present disclosure, can make and use such trapezoidal seals using no more than routine experimentation.

In some embodiments, the cushion 214 is formed by drawing a first thermopolymer sheet portion into a molding apparatus with heat and vacuum to form the skin contact face and the third face. A molding apparatus is, in its simplest form, a vacuum mold having a cavity or well in the shape of the skin contact face and third face. In a first step, a polymer sheet may be heated and drawn under vacuum to form the exterior bladder boundary of the skin contact face and the third face. The vacuum may be created through the use of air holes in the bottom of the mold cavity. Because the skin contact face and the third face meet at the bottom of the heated vacuum cavity, the transition between those faces may be rounded, thereby enabling removal of the molded product and generally facilitating the process. A gel, foam, liquid rubber or other like viscous material may then be poured into the mold to fill the cavity formed by the first polymer sheet. A second polymer sheet (or another portion of the same polymer sheet) may then be overlaid on the viscous material-filled cavity and the edges of the two polymer sheets may be heat-bonded to seal the viscous material within the cushion 214.

When drawn under heat and vacuum, the drawn thermopolymer sheet portion(s) will be made thinner than the starting stock material. One of skill in the art will recognize the effects of vacuum drawing on thermopolymer sheet thickness. For particular applications, one of skill in the art may select polymer sheets having varying thicknesses. For example, a thinner film will maximize the ability for the filled cushion to contour. Thicker films are often used for deeper draws in the heated mold.

In some embodiments, the cushion 214 may be configured with one or more local lobes positioned at locations correspond with areas of dramatic feature change in the contour of a face to be fit (e.g., around the nose and the bridge of the nose). To enable inclusion of the one or more local lobes, the cavity of the molding apparatus may contain a local cavity (i.e., a cavity deeper than the cavity forming adjacent non-lobe portions) deepening the draw to form the one or more local lobes. A local lobe may have an upper portion and a lower portion. The upper portion may be both local and prominent, rising dramatically from the non-lobe portion of the cushion 214. The upper portion may be trapezoidal in shape based on the manufacturing process.

The side projection of a local lobe can be broken down into a generally rectangular, lower portion, the height of which may approximate the height of side projections of adjacent non-lobe portions of the cushion 214, and a generally trapezoidal portion sitting atop the rectangular portion. The trapezoidal portion represents the local lobe rising dramatically from the adjacent non-lobe portion of the cushion 214. The angled side legs of the trapezoid may form an average angle with the top of the rectangular portion and the average angle may be between 30 degrees and 89 degrees.

In designing the local lobes, one of skill in the art will recognize and consider several design factors. The lobe should be large enough to seal around features of the face with dramatic structural changes. An example given previously is the nose and the bridge of the nose. It will also be recognized that lobes that are too large will also result in a “leaky” seal caused by the upper portion of the local lobe lifting the adjacent non-lobe portion off the face.

The cushion 214 may comprise one or more local lobes extending the skin contact face and the third face for sealing areas of substantial variation in facial structure of the user. For example, on a facemask that covers the nose and mouth, local lobes may be located at the portion of the cushion 214 that positions on either side of the nose and the bridge of the nose to compensate for the extreme change in contour of the face at this position. Likewise, local lobes may be advantageous on the cushion 214 when the facemask is a nasal facemask or one covering the eyes, as well as the nose and mouth, as areas of substantial variation in facial structure can vary depending on the type of facemask used. For example, when the facemask body 212 is that of a nasal facemask, local lobes may be beneficial on either side of the nose and the bridge of the nose. Moreover, when the facemask body 212 is that of a facemask covering the eyes, local lobes may be beneficially located adjacent to the eye sockets, under the lower lip or at the cheeks.

The cushion 214, when configured with the three primary external faces, is designed to form a seal between the skin contact face and the face of the user. As contact is made, the third face bulges and the skin contact face “lays down” increasing the contact area with the face. The triangular shape of the seal of the present disclosure allows for the skin contact face to “lay down” (or redirect) allowing the majority of the surface of the plane to make contact and remain in contact with the user's face. As discussed above, the modified prior art semi-circular sealing element of the prior art forms an inferior seal.

Examples of the cushion 214 are those described in U.S. Pat. No. 10,974,007, the contents of which are hereby incorporated by reference in their entirety.

### Mask Kits

The present disclosure provides various facemask kits.

In some embodiments, a facemask kit may include the facemask body 212 having the cushion 214 attached thereto and the third frame member 202 decoupled from the facemask body 212.

In some embodiments, a facemask kit may include the facemask body 212 having the cushion 214 attached thereto as well as the third frame member 202 coupled to the facemask body 212.

### Further Definitions and Construction of Terms

The titles, headings, and subheadings provided herein should not be interpreted as limiting the various aspects of the disclosure. Accordingly, the terms defined herein are more fully defined by reference to the specification in its entirety. All references cited herein are incorporated by reference in their entirety.

Unless otherwise defined, scientific and technical terms used herein shall have the meanings that are commonly understood by those of ordinary skill in the art. Further, unless otherwise required by context, singular terms shall include pluralities, and plural terms shall include the singular.

In this application, the use of “or” means “and/or” unless stated otherwise. In the context of a multiple dependent claim, the use of “or” refers back to more than one preceding independent or dependent claim in the alternative only.

It is further noted that, as used in this specification and the appended claims, the singular forms “a,” “an,” and “the,” and any singular use of any word, include plural referents unless expressly and unequivocally limited to one referent.

As used herein, the term “about,” means approximately. When the term “about” is used in conjunction with a numerical range, it modifies that range by extending the boundaries above and below the numerical values set forth. Illustratively, the use of the term “about” indicates that values slightly outside the cited values (i.e., plus or minus 0.1% to 10%), which are also effective and safe are included in the value. Numerical ranges recited herein by endpoints include all numbers and fractions subsumed within that range (e.g. 1 to 5 includes 1, 1.5, 2, 2.75, 3, 3.90, 4, and 5).

As used herein, the terms “comprising” (and any form of comprising, such as “comprise,” “comprises,” and “comprised”), “having” (and any form of having, such as “have” and “has”), “including” (and any form of including, such as “includes” and “include”), and “containing” (and any form of containing, such as “contains” and “contain”) are inclusive or open-ended and do not exclude additional, un-recited elements or method steps. Additionally, a term that is used in conjunction with the term “comprising” is also understood to be able to be used in conjunction with the term “consisting of” or “consisting essentially of.”

Method steps described in this disclosure can be performed in any order unless otherwise indicated or otherwise clearly contradicted by context.

For the avoidance of doubt, insofar as is practicable any embodiment of a given aspect of the present disclosure may occur in combination with any other embodiment of the same aspect of the present disclosure. In addition, insofar as is practicable it is to be understood that any preferred or optional embodiment of any aspect of the present disclosure should also be considered as a preferred or optional embodiment of any other aspect of the present disclosure.

What is claimed is:

1. A headgear frame member, comprising:
a first elongated portion having a first terminal end portion comprising a first coupling mechanism for removably coupling to headgear;
a second elongated portion oriented non-parallel to the first elongated portion, the second elongated portion having a second terminal end portion comprising a second coupling mechanism for removably coupling to the headgear; and
a third elongated portion oriented non-parallel to the first elongated portion and the second elongated portion,
wherein the third elongated portion has an axis extending along the third elongated portion,
wherein the first elongated portion extends a first distance along the axis,
wherein the second elongated portion extends a second distance along the axis, and
wherein the first distance is different than the second distance.

2. The headgear frame member of claim 1, wherein the first distance is greater than the second distance.

3. The headgear frame member of claim 1, wherein:
the third elongated portion has a third terminal end portion;
the first coupling mechanism is a third distance away from the third terminal end portion along the axis; and
the second coupling mechanism is a fourth distance away from the third terminal end portion along the axis.

4. The headgear frame member of claim 3, wherein the third distance is greater than the fourth distance.

5. The headgear frame member of claim 3, wherein:
the third terminal end portion comprises a third coupling mechanism;
the first coupling mechanism is the third distance away from the third coupling mechanism along the axis; and
the second coupling mechanism is the fourth distance away from the third coupling mechanism along the axis.

6. The headgear frame member of claim 5, wherein the third distance is greater than the fourth distance.

7. The headgear frame member of claim 1, wherein:
the first coupling mechanism comprises a first elongated aperture having a first major axis;
the second coupling mechanism comprises a second elongated aperture having a second major axis; and
the first major axis and the second major axis intersect at a location between the headgear frame member and the back of the user's head.

8. The headgear frame member of claim 1, wherein:
the first coupling mechanism removably couples to the headgear above an ear of a user; and
the second coupling mechanism removably couples to the headgear below the ear of the user.

9. The headgear frame member of claim 1, wherein the third elongated portion has a third terminal end portion comprising a third coupling mechanism for removably coupling to another headgear frame member.

10. The headgear frame member of claim 9, wherein the third coupling mechanism comprises a hook member sized to removably couple to the perimeter of an aperture in the other headgear frame member.

11. The headgear frame member of claim 1, wherein the first and second elongated portions are positioned at an angle of about 60° to 90° with respect to each other.

12. The headgear frame member of claim 11, wherein the angle is about 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, or 90°.

13. The headgear frame member of claim 12, wherein the angle is about 80°.

14. The headgear frame member of claim 1, wherein when the hypotenuse of a right triangle is positioned along a portion of the headgear removably coupled to the first elongated portion, the interior angle of the corner of the right triangle abutting the portion of the headgear is about 25° to 55°, and the interior angle of the corner of the right triangle abutting the first elongated portion is about 35° to 65°.

15. The headgear frame member of claim 14, wherein the interior angle of the corner of the right triangle abutting the portion of the headgear is about 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, or 55°.

16. The headgear frame member of claim 15, wherein the interior angle of the corner of the right triangle abutting the portion of the headgear is about 40°.

17. The headgear frame member of claim 14, wherein the interior angle of the corner of the right triangle abutting the first elongated portion is about 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, or 65°.

18. The headgear frame member of claim 17, wherein the interior angle of the corner of the right triangle abutting the first elongated portion is about 50°.

19. A facemask kit, comprising:
the headgear frame member of claim 1; and
headgear comprising a first elongated member and a second elongated member,
wherein the first elongated member of the headgear removably couples to the first elongated portion of the first frame member and adjustment of the tension of the first elongated member alters the force exerted perpendicularly with respect to a sealing element of the facemask, and
wherein the second elongated member of the headgear removably couples to the second elongated portion of the first frame member and adjustment of the tension of the second elongated member vertically raises and lowers the positioning of the facemask on the user's face.

20. The facemask kit of claim 19, further comprising a second headgear frame member comprising:
a third elongated portion having a third terminal end portion comprising a third coupling mechanism for removably coupling to the headgear;
a fourth elongated portion oriented non-parallel to the third elongated portion, the fourth elongated portion having a fourth terminal end portion comprising a fourth coupling mechanism for removably coupling to the headgear; and
a fifth elongated portion oriented non-parallel to the third elongated portion and the fourth elongated portion,
wherein the fifth elongated portion has a second axis extending along the fifth elongated portion,
wherein the third elongated portion extends the first distance along the second axis, and
wherein the fourth elongated portion extends the second distance along the second axis.

21. The facemask kit of claim 20, further comprising a third headgear frame member that removably couples to the headgear frame member and the second headgear frame member.

22. The facemask kit of claim 21, further comprising a facemask body that removably couples to the third headgear frame member.

23. The facemask kit of claim 22, wherein the facemask body comprises a fluid-filled cushion that facilitates a seal between the facemask body and the user's face.
